(19) United States
(12) Patent Application Publication
(10) Pub. No.: US 2025/0089732 A1
(43) Pub. Date: Mar. 20, 2025

(54) PURIFIED HUMAN MILK OLIGOSACCHARIDES COMPOSITIONS

(71) Applicant: Prolacta Bioscience, Inc., Duarte, CA (US)

(72) Inventors: Adam SUN, Duarte, CA (US); Annabelle LE BOUEDEC, Duarte, CA (US); Tin D. HUYNH, Duarte, CA (US); Kim Thu TRAN, Duarte, CA (US)

(21) Appl. No.: 18/406,627

(22) Filed: Jan. 8, 2024

Related U.S. Application Data
(63) Continuation of application No. 16/334,167, filed on Mar. 18, 2019, now abandoned.
(60) Provisional application No. 62/396,779, filed on Sep. 19, 2016.

(51) Int. Cl. A23C 9/142; A23C 9/12; A23C 9/20; A23L 33/125; A61K 31/702; A61K 35/20; A61P 37/06

(52) U.S. Cl. CPC A23C 9/1422; A23C 9/1206; A23C 9/206; A23L 33/125; A61K 31/702; A61K 35/20; A61P 37/06

(57) ABSTRACT

The present invention relates to purified and concentrated human milk oligosaccharide compositions and methods of making and using the same.

### CROSS REFERENCE TO RELATED APPLICATIONS

This application claims priority to U.S. Provisional Application No. 62/396,799 filed on Sep. 19, 2016, the contents of which are hereby incorporated by reference in its entirety.

### FIELD OF THE INVENTION

The invention relates to a process for producing substantially purified human milk oligosaccharide (HMO) compositions, the substantially purified compositions produced thereby as well as methods for using the compositions.

### BACKGROUND OF THE INVENTION

Human milk oligosaccharides (HMOs) are a family of structurally diverse unconjugated glycans that are highly abundant in and unique to human milk. Originally, HMOs were proposed to be prebiotic “bifidus factors,” or human milk glycans found to promote growth in Bifidobacterial species of the gut and found uniquely in the stool of breast fed infants compared to formula fed infants. Additional studies suggested that diverse milk glycans are responsible, in part, for the health benefits associated with breast feeding. Today, HMOs are known to be more than just “food for bugs.” An accumulating body of evidence suggests that HMOs are antiadhesive antimicrobials that serve as soluble decoy receptors preventing pathogen attachment to infant mucosal surfaces and thereby lowering the risk for viral, bacterial and protozoan parasite infections. In addition, HMOs are thought to modulate epithelial and immune cell responses, thereby reducing excessive mucosal leukocyte infiltration and inflammation, thereby, lowering the risk of necrotizing enterocolitis as well as providing the infant with sialic acid as a potentially essential nutrient for brain development and cognition.

HMOs are composed of the five monosaccharides glucose (Glc), galactose (Gal), N-acetylglucosamine (GlcNAc), fucose (Fuc) and sialic acid (Sia), with N-acetylneuraminic acid (Neu5Ac) as the predominant if not only form of Sia. More than two hundred different HMOs have been identified so far, but not every woman synthesizes the same set of oligosaccharides nor in the same amounts (reviewed in Kobata 2010). Therefore, the population diversity for HMOs is often much greater than that of any one woman.

What is more, composition and concentration of oligosaccharides also vary over the course of lactation (reviewed in Kunz et al. 2000). Colostrum contains as much as 20-25 g/L of HMO, however, as milk production matures, total HMO concentrations decline to 5-20 g/L often still exceeding the concentration of total milk protein, making the HMO fraction of human milk the third most abundant fraction after lactose and fat. The wide range in HMO concentration and diversity reported for HMO reflects not only known genetic variations in glycosylation pathways among women, but also technical differences in the analytical methods used in the detection and quantitation of HMO by various academic and contract research laboratories.

What is clear, however is that the oligosaccharides present in the milk of other mammals, such as cows, sheep and goats are much less abundant and structurally distinct than oligosaccharides in human milk. For example, even the most oligosaccharide-rich portion of bovine milk, colostrum, only contains approximately 50 molecular species of oligosaccharides. Goats milk, which is thought to contain the most structurally analogous milk oligosaccharide profile to the HMOs, contains only about 40 molecular species, less than 25% of the characterized diversity of HMOs (Thum, et al. 2015)

In addition to limitations on the availability of raw material, another major impediment to the production of a milk oligosaccharide composition is the reduction of lactose and other minerals which tend to concentrate with the oligosaccharide portions of milk during their isolation and concentration, particularly when ultrafiltration, as opposed to protein precipitation, is used to remove protein and to do so without loss of yield. While this remains a process limitation regardless of the species of milk being processed, nowhere is this problem felt more acutely than in the preparation of a human oligosaccharide composition, since the starting material is so scarce making loss of yield unacceptable.

Others have attempted to solve this problem by using solvent-based systems to remove protein and other macronutrients. This method prevents the accumulation of lactose and minerals associated with the ultrafiltration process. In fact, it has been reported that this method can actually aid in the removal of lactose (See e.g. Sarney, 2000). This process, however, requires the use of solvents and effectively destroys the remainder of the human milk rendering it unavailable to be used for other lifesaving products. With a commodity as scarce as human milk, this is simply unacceptable.

The ultrafiltration process used to generate human milk permeate, as used herein, while avoiding the use of potentially harmful organic solvents and saving the protein fraction to use in other lifesaving products, only exacerbates the problem of lactose and minerals in milk. The lactose content of concentrated human milk permeate, for example, may be as high as 10-15% in some instances, compared to lactose levels of ≤6%, the concentration found in milk. These levels of lactose are difficult to digest, even for people who are enzymatically capable of digesting lactose, to say nothing of those that are not. Several approaches have been used to remove lactose including enzymatic digestion followed by serial diafiltration to remove the enzyme used for digestion. Even in these samples in which protein was removed by precipitation with an organic solvent, as opposed to ultrafiltration which concentrates lactose and minerals, a significant level of lactose remains following diafiltration to say nothing of the mineral content of this composition. (See e.g. Sarney, 2000 and Grandison, et al 2002) What is more, diafiltration of HMO compositions also results in the unacceptable loss of low molecular weight HMO species, for example, 2FL.

Since there are no natural resources available to provide access to large amounts of purified HMO, most infant formulas on the market provide neonates with no oligosaccharides whatsoever, and those that do provide either non-naturally occurring oligosaccharides meant to mimic HMO, including galactooligosaccharides (GOS) and fructooligosaccharides (FOS) or, more recently, chemically synthesized versions of the naturally occurring HMOs, LNnT and 2′-FL (Bode, 2015). While these compositions may represent improvements to completely HMO-free compositions, they are substantially less diverse with respect to the molecular species of HMO than the average human milk and certainly much less diverse than human milk when you look across the population.

What is needed is a process that allows for the efficient recovery, concentration and purification of an HMO composition that is structurally and functionally diverse, but with a substantially reduced lactose and/or mineral content.

### SUMMARY OF THE INVENTION

Provided herein are methods of manufacturing human milk oligosaccharide compositions that retain the structural and functional diversity of the oligosaccharides found across the population of human milk while having substantially reduced lactose and/or mineral concentrations. The methods provided herein have the advantage of being scalable and the added advantage of not destroying the remaining milk fractions, for example by the use of solvents to remove protein.

In one embodiment, a method for making a purified human milk oligosaccharide (HMO) composition is provided. In one embodiment, the method includes mixing a human milk permeate with an enzyme capable of digesting lactose under conditions suitable for digestion of the lactose in the permeate and for a period of time sufficient for such digestion. In some embodiments, the enzyme is a lactase enzyme. In some embodiments, the lactase enzyme is removed from the lactase digested permeate mixture after digestion. In some embodiments, prior to lactase removal, the permeate/lactase mixture is clarified, for example, through depth filters. In some embodiments, the lactase is removed from the mixture by filtration. In some embodiments, the filtration comprises filtration through a membrane with a pore size of about 50,000 Dalton. In some embodiments, the method further comprises filtering the mixture through one or more additional filters. In one embodiment, the one or more additional filters comprises a membrane with a pore size of about 2,000 to about 3,000 Dalton. In one embodiment, the one or more additional filters comprises a membrane with a pore size of about 600 Dalton.

In some embodiments, prior to or concurrent with the addition of the lactase enzyme to the permeate, the pH and/or heat of the permeate is adjusted. In one embodiment, the pH is adjusted to about 4.3 to about 4.7. In one embodiment, the pH is adjusted to about 4.5. In one embodiment, the heat of the permeate mixture is adjusted prior to or concurrent with the addition of the lactases. In one embodiment, the heat is adjusted to a temperature of about 45° C. to about 55° C. In one embodiment, the heat is adjusted to a temperature of about 50° C. In one embodiment, the pH of the permeate is adjusted to about 4.3 to about 4.7 and the heat is adjusted to a temperature of about 45° C. to about 55° C.

In one embodiment, the lactases is added at a concentration of about 0.1% to about 0.5% w/w. In some embodiments, the lactase is added at a concentration of about 0.1% w/w. In some embodiments, the lactase is incubated with the permeate for about 5 to about 225 minutes. In some embodiments, the lactase is incubated with the permeate for about 15 to about 120 minutes. In some embodiments, the lactases is incubated with the permeate for about 30 to about 90 minutes. In some embodiments, the lactase is incubated with the permeate for about 60 minutes.

In one embodiment, after incubation, the permeate/lactase mixture is cooled to a temperature of about 20° C. to about 30° C. In one embodiment, the permeate/lactase mixture is cooled to a temperature of about 25° C. In one embodiment, the permeate/lactase mixture is clarified. In one embodiment, the permeate/lactase mixture is clarified through a depth filter. In one embodiment, the depth filter comprises a filter of about 1 micron to about 5 microns.

In one embodiment, the lactase is removed via filtration. In one embodiment, the lactase is removed via filtration through a filter with a pore size of about 50,000 Daltons. In one embodiment, the composition is further filtered through one or more additional filters. In some embodiments, the one or more additional filters comprises a membrane with a pore size of about 2,000 to about 3,000 Daltons. In some embodiments, the one or more additional filters comprises a membrane with a pore size of ≤600 Daltons. In some embodiments, the composition is filtered through both a filter comprising a membrane of about 2,000 to about 3,000 Daltons followed by filtration through a membrane of ≤600 Daltons.

In some embodiments, purified HMO compositions made by the methods of the current invention are provided. In some embodiments, the purified HMO composition has a reduced level of lactose and minerals compared to permeate. In some embodiments, the purified HMO composition comprises less than about 5.0% w/w lactose. In some embodiments, the HMO composition comprises the mineral profile of Table 1. In one embodiment, the purified HMO composition comprises an HMO concentration of about 0.5% to about 7.5% HMO. In some embodiments, the purified HMO composition comprises an HMO concentration of about 1.0% to about 2.0% HMO. In some embodiments, the purified HMO composition comprises an HMO concentration of about 2.0% to about 4.0% HMO. In some embodiments, the purified HMO composition comprises an HMO concentration of about 4.0% to about 5.0% HMO. In some embodiments, the purified HMO composition comprises an HMO concentration of about 5.0% to about 7.5% HMO. In some embodiments, the purified HMO composition comprises an HMO concentration of about 5.0% w/w HMO. In one embodiment, the HMO profile made according to the methods described herein comprises the HMO profile as shown in FIGS. 5 (E and F).

In some embodiments, provided herein are methods for administering the purified HMO composition to a subject in need thereof. In some embodiments, provided herein is a method for treating or preventing NEC in a subject in need thereof. In some embodiments, a method for decreasing systemic inflammation is provided by administering the purified HMO composition made by the methods described herein. In some embodiments, a method for treating or preventing infection in a subject in need thereof is provided. In some embodiments, a method for treating or preventing a viral or bacterial infection by administering the purified HMO composition made by the methods described herein is provided. In some embodiments, the bacterial infection is a Clostridium difficile infection. In some embodiments, the viral infection is a norovirus or a rotavirus.

In some embodiments, the purified HMO composition is administered before, during or after an additional pharmaceutical or therapeutic agent. In some embodiments, the purified HMO composition is administered before during or after a fecal, organ or bone marrow transplant. In some embodiments, the purified HMO composition is administered before during or after an antibiotic, antiviral, or antifungal treatment regimen. In some embodiments, the purified HMO composition is administered before during or after a probiotic composition. In some embodiments, the purified HMO composition is administered before during or after chemotherapy and/or radiation.

### BRIEF DESCRIPTION OF THE DRAWINGS

FIG. 1 shows a schematic of an exemplary HMO production process.

FIG. 2 shows a schematic of an alternative HMO production process.

FIG. 3 shows a schematic of the process used to produce 20× concentrated permeate from ≥8× concentrated permeate from ≥8× concentrated permeate.

FIG. 4 shows (A) a schematic of the process used to formulate the purified HMO composition and (B) the process used to pasteurize and fill the purified HMO composition

FIGS. 5A-5F show the results of HPAEC-PAD chromatography of neutral (FIGS. 5A, 5C, and 5E) and sialylated (FIGS. 5B, 5D, and 5F) HMOs from pooled donor milk (FIGS. 5A and 5B), human milk permeate (FIGS. 5C and 5D), and purified HMO compositions (FIGS. 5E and 5F).

FIG. 6 shows the global untargeted metabolomics of serum, feces and urine from adults administered an HMO obtained using LC/MS/MS and Polar LC. Results show parenteral HMO and HMO breakdown products detected in (A) serum, (B) urine, (C) feces and (D) milk.

FIG. 7 shows (A) the metabolic pathway of eicosanoids obtained using LC/MS/MS and Polar LC and (B and C) the levels of the eicosanoid metabolites over time in subjects ingesting the purified HMO compositions made by the methods of the invention.

FIG. 8 shows the serum levels of sphingolipid metabolites using LC/MS/MS and Polar LC over time in subjects ingesting the purified HMO compositions made by the methods of the invention.

### DETAILED DESCRIPTION

The present invention provides processes for producing purified human milk oligosaccharide compositions that have substantially reduced lactose and mineral content, the novel compositions produced thereby as well as methods for using such novel compositions. The process begins with filtered portions of pooled human milk, therefore the purified HMO compositions of the present invention can contain a more diverse profile of discrete molecular species of HMO compared to any typical individual woman. Thus, the compositions herein are often said to be representative of the population of HMOs, which is in contrast to being representative of an individual person's HMO profile.

By “human milk oligosaccharide(s)” (also referred to herein as “HMO(s)”) is meant a family of structurally diverse unconjugated glycans that are found in human breast milk.

Human milk oligosaccharides are carbohydrates that contain lactose at the reducing end and, typically, a fucose or a sialic acid at the non-reducing end (Morrow et al. 2005). These terminal sugars are the residues that most strongly influence the selective growth of bacteria and the interaction of oligosaccharides with other molecules or cells, including bacterial pathogens in the gut lumen. Furthermore, sialic acids are structural and functional components of brain gangliosides and have been implicated in neurological development of infants.

Oligosaccharides can be free or conjugated as glycoproteins, glycolipids etc. and are classified as glycans. They constitute the third most numerous solid component of human milk, after lactose and lipid (Morrow, 2005). The majority of milk oligosaccharides, however, are not digestible by infants and can be found in infant feces largely intact.

By “permeate” is meant a portion of milk (e.g. pooled human milk) that is the product of ultrafiltration. Specifically, the liquid that is left after the ultrafiltration (e.g. through a filter of about 1-1000KDa). The liquid that passes through this ultrafiltration process is referred to as permeate. The retentate of this process concentrates human milk protein which may then be used to create other life-saving formulations, for example, to make human milk fortifier compositions, such as those described in, U.S. Pat. No. 8,377,455. Thus, in contrast to methods that rely on protein precipitation with solvents, which may contaminate the HMO product, the use of ultrafiltration to obtain a substantially protein-free starting material as used herein, preserves the remainder of the valuable macronutrients in human milk while avoiding the use of organic solvents.

By “milk” is meant the fluid that is produced by the mammary gland of a mammal and expressed by the breast. Milk includes all lactation products including, but not limited to colostrum, whole milk and skim milk taken at any point post parturition. Unless otherwise specified, as used herein “milk” refers typically to whole human milk.

By “whole milk” is meant milk (e.g. pooled human milk) from which no fat has been removed.

By “skim milk” is meant milk (e.g. pooled human milk) from which at least 75% of fat has been removed or alternatively, milk that has been subject to centrifugation to remove the fat.

By “substantially” as in “substantially reduced lactose- and/or mineral content” is meant that the reduction in the level of minerals and/or lactose represents a statistical difference when compared to concentrated permeate that has not been subject to the current methods. By way of example, in some embodiments, the purified HMO compositions with substantially reduced lactose comprise lactose levels of ≤5%.

By “consisting essentially” of, as used herein refers to compositions containing particular recited components while excluding other major bioactive factors. For example, a composition consisting essentially of HMOs, would exclude such things as protein, fat, exogenously added material, but may contain other inert or trace material, such as water, acceptable levels of certain salts, microRNAs, or exosomes, for example.

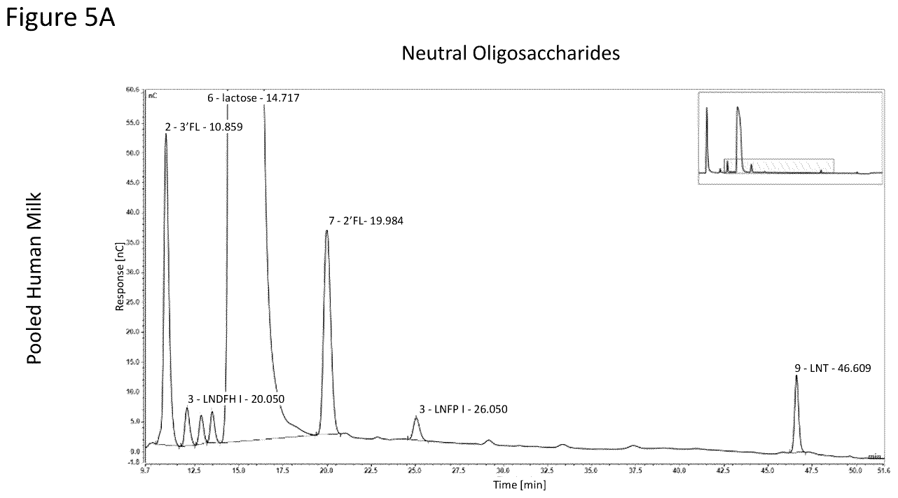

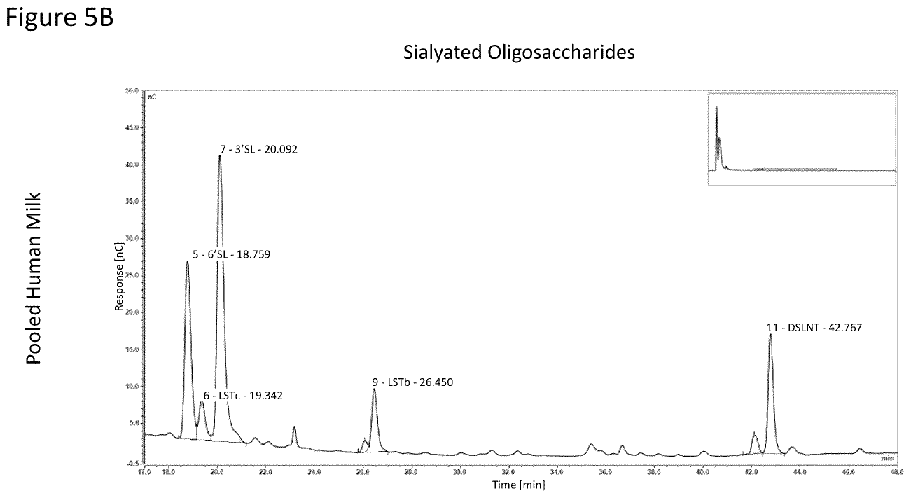

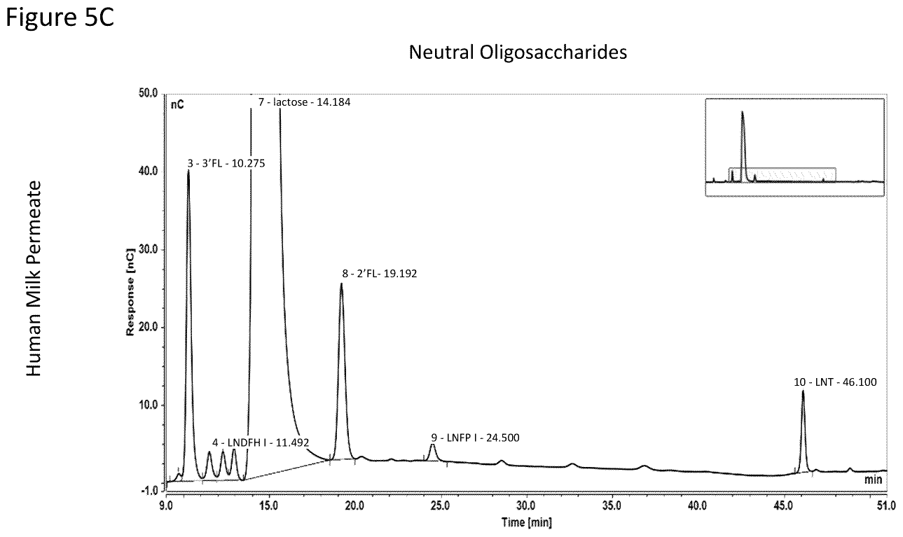

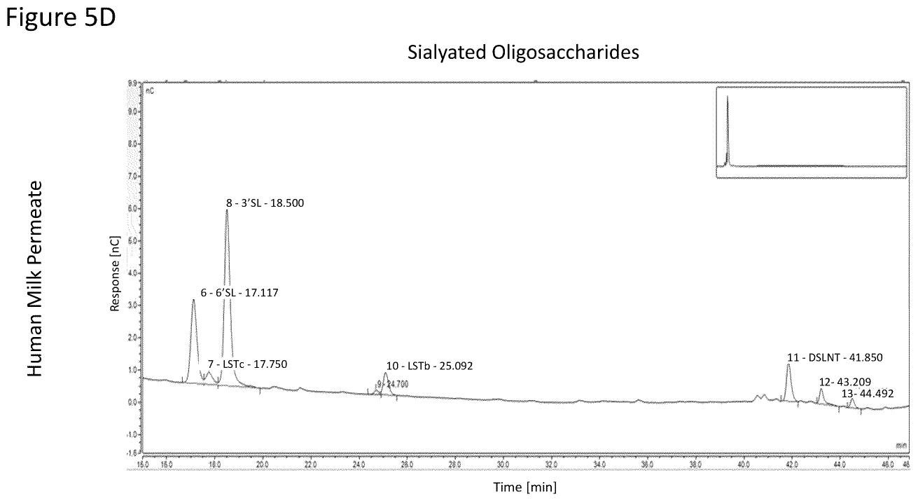

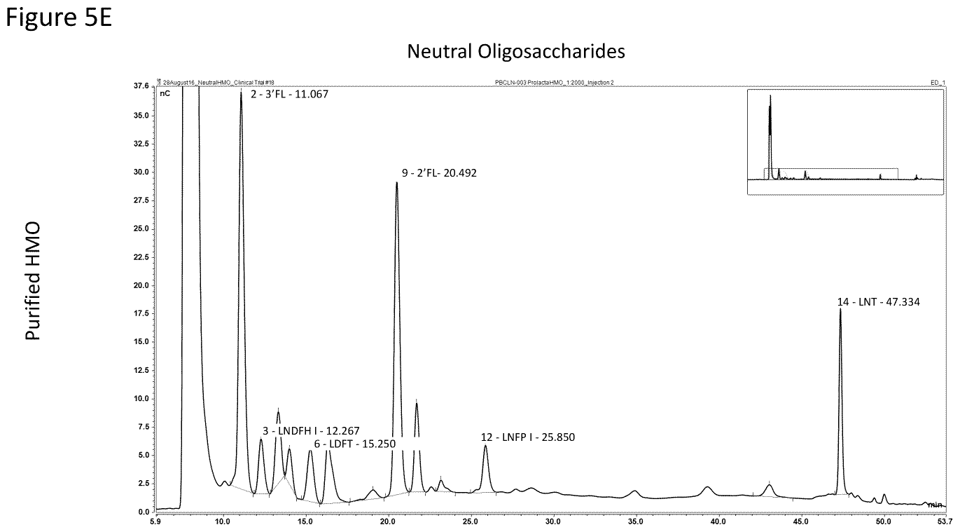

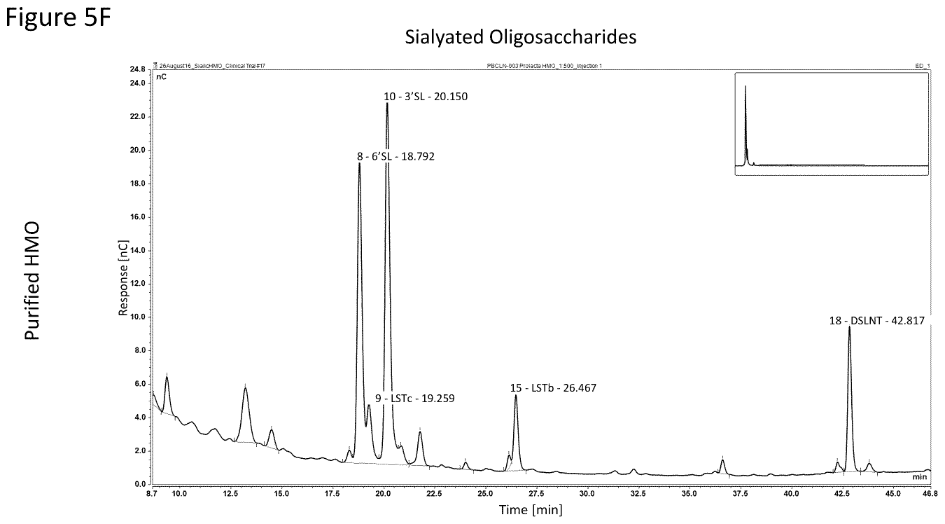

The term “purified HMO composition” as used herein is meant an HMO composition (e.g. a concentrated human permeate) with substantially reduced levels of lactose and/or minerals and produced by the methods provided herein. An exemplary purified HMO composition is depicted in FIGS. 5 (E) and (F).

### Methods of Making Purified HMO Compositions

Human milk permeate serves as the starting material from which the purified HMO compositions of the present invention are produced by the processes described herein. Methods for obtaining human milk permeate can be found, for example in U.S. Pat. No. 8,927,027, which is incorporated by reference herein in its entirety.

Briefly, pooled milk from pre-qualified donors that has been screened for drugs, contaminants, pathogens, and adulterants and filtered to remove heat resistant bacterial spores is separated (e.g. by centrifugation) into cream and skim fractions. The skim fraction undergoes further filtration, e.g., ultrafiltration, e.g., with a pore size between 1-1000 kDa to obtain a protein rich retentate and the HMO-containing permeate. Details of this process can be found, for example, in U.S. Pat. Nos. 8,545,920; 7,914,822; 7,943,315; 8,278,046; 8,628,921; and 9,149,052, each of which is hereby incorporated by reference in its entirety.

In one embodiment, a process for producing a purified HMO composition with substantially reduced levels of lactose is provided. This process requires the biochemical and/or enzymatic removal of lactose from the lactose-rich human milk permeate fraction, without loss of yield or change in molecular profile of the HMO content of human milk permeate. And, in some embodiments, without leaving residual inactivated foreign protein, if enzymatic digestion is used to reduce lactose.

In one embodiment, the process for reducing lactose from human milk permeate, and therefore from the purified HMO composition comprises the steps of a) adjusting the pH of the permeate mixture; b) heating the pH adjusted mixture; c) adding lactase enzyme to the heated permeate mixture to create a permeate/lactase mixture and incubating a period of time; d) removing the lactase from the mixture and filtering the mixture to remove lactase; and e) concentrating human milk oligosaccharides. While the steps described here are listed in chronological order, one of skill in the art would understand that the order in which steps (a)-(c) are performed may be varied. That is to say, and by way of example only, the lactase enzyme may be added prior to heating the mixture, or, alternatively at any point during the heating process. Similarly, and also by way of example only, the mixture may be heated prior to adjustment of the pH. Furthermore, several steps may be grouped into a single step, for example “enzymatically digesting lactose” or “lactases digestion of lactose” involves steps (a)-(c) as described, supra. These steps may be performed concurrently or consecutive in any order. Therefore, as used herein “lactose digestion” refers to the performance of at least these three steps, in any order, consecutively or concurrently.

In one embodiment, the pH of the permeate is adjusted to a pH of about 3 to about 7.5 In one embodiment, the pH is adjusted to a pH of about 3.5 to about 7.0. In another embodiment, the pH is adjusted to a pH of about 3.0 to about 6.0. In yet another embodiment, the pH is adjusted to a pH of about 4 to about 6.5. In yet another embodiment, the pH is adjusted to a pH of about 4.5 to about 6.0. In still another embodiment, the pH is adjusted to a pH of about 5.0 to about 5.5. In still another embodiment, the pH is adjusted to a pH of about 4.3 to about 4.7, preferably 4.5. The pH may be adjusted by adding acid or base. In some aspects, pH is adjusted by adding acid, for example HCl. In yet other aspects, pH is adjusted by adding IN HCl and mixing for a period of time e.g. about 15 minutes.

In one embodiment, the pH-adjusted permeate is heated to a temperature of about of about 25° C. to about 60° C. In another embodiment, the permeate is heated to a temperature of about 30° C. to about 55° C. In another embodiment, the permeate is heated to a temperature of about 40° C. to about 50° C. In another embodiment, the permeate is heated to a temperature of about 48° C. to about 50° C. In yet another embodiment, the permeate is heated to a temperature about 50° C. In yet another embodiment, the permeate is heated to a temperature less than or equal to about 40° C.

In one aspect, lactase enzyme is added to the pH-adjusted, heated permeate to create a permeate/lactase mixture and in order to break down lactose into monosaccharides. In one embodiment, lactase enzyme is added at about 0.1% w/w to about 0.5% w/w concentration. In yet another aspect, lactase enzyme is added at about 0.1% w/w, or 0.2% or 0.3% or 0.4% or 0.5% w/w. There are many commercially available lactase enzymes that may be used. As such, the lactase enzyme may be derived from any origin (e.g. fungal or bacterial in origin).

In some embodiments, the pH-adjusted, heated permeate is incubated with the lactase enzyme for about 5 to about 225 minutes. In some embodiments, the incubation time is about 15 min to about 90 min. In some embodiments, the incubation time is about 30 minutes to about 90 minutes. In some embodiments, the incubation time is about 60 minutes. One of skill in the art will understand that incubation time is dependent upon myriad of factors including, but not limited to, the source of the enzyme used, the temperature and pH of the mixture and the concentration of enzyme used. Any of these variables may require a longer or shorter incubation time with the lactase enzyme. While the pH, temperature, and enzyme incubation conditions provided here are what work optimally for the process described herein, one of skill in the art would understand that modifications may be made to one or more of these variables to achieve similar results. For example, if less enzyme is used than the about 0.1% w/w to about 0.5% w/w described herein, the incubation time may need to be extended to achieve the same level of lactose digestion. Similar adjustments may be made to both the temperature and pH variables as well.

In one embodiment, after incubation the permeate/lactase mixture is cooled to a temperature of about 20° C. to about 30° C. In a particular embodiment, the permeate/lactase mixture is cooled to a temperature of about 25° C.

In one embodiment, the permeate/lactase mixture is clarified to remove insoluble constituents. In certain instances, insoluble material may form throughout the change in pH and temperature. Therefore, in some embodiments, it may be necessary or beneficial to clarify the mixture to remove these insoluble constituents, for example, through a depth filter. The filters may be 0.1 to 10 micron filters. In some embodiments, the filters are about 1 to about 5 micron filters. Alternatively, removal of insoluble constituents can be achieved through a centrifugation process or a combination of centrifugation and membrane filtration. The clarification step is not essential for the preparation of a diverse HMO composition, as described herein, rather, this optional step aids in obtaining a more purified HMO composition. Furthermore, the clarification step is important in the reusability of the filtration membranes and thus to the scalability of the process. Without adequate clarification, one will require substantially more filter material making it difficult and expensive to produce HMO compositions at clinical scale. However, one will understand that more or less stringent clarification may be formed at this stage in order to produce more or less purified HMO compositions, depending on formulation and application. For example, precipitated minerals may be less of a problem for a formulation destined for lyophilization or formulations destined for use in healthy adults compared to a liquid formulation or formulations for use in fragile populations (e.g. neonates).

Furthermore, it may be desirable in some instance to remove the spent and excess lactase enzyme from the clarified permeate/latctase mixture. There may, however, be some instances where the inactivated foreign protein will carry no biological risk and therefore the added steps of lactase removal or even inactivation may not be necessary. In some embodiments, the spent and excess lactase is inactivated, for example by high temperature, pressure or both. In some embodiments, the inactivated lactase is not removed from the composition.

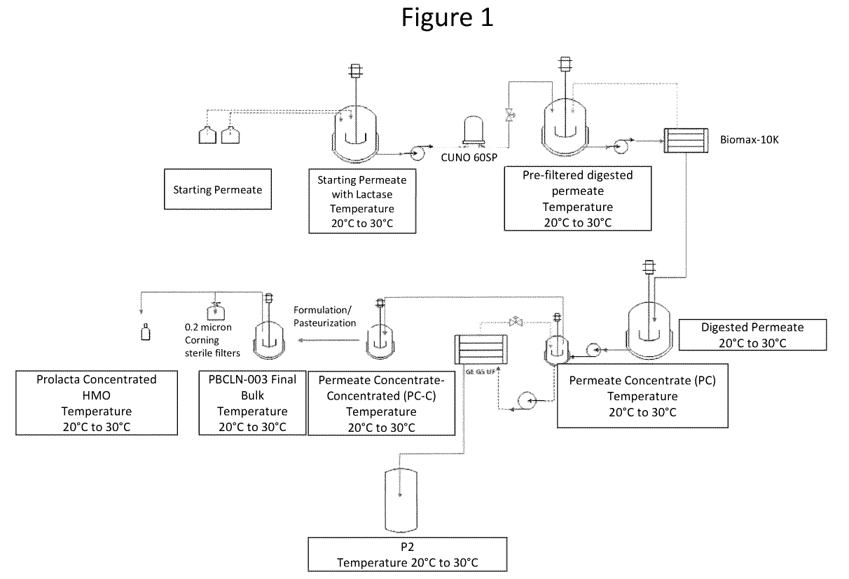

In other embodiments, however, a further purification to remove foreign proteins will be called for. In such embodiments lactase enzyme removal may be accomplished by ultrafiltration. In some embodiments, ultrafiltration is accomplished using an ultrafiltration membrane, for example using a membrane with molecular weight cut-off of ≤50,000 Dalton, e.g. a BIOMAX-50K. (See e.g. FIG. 1)

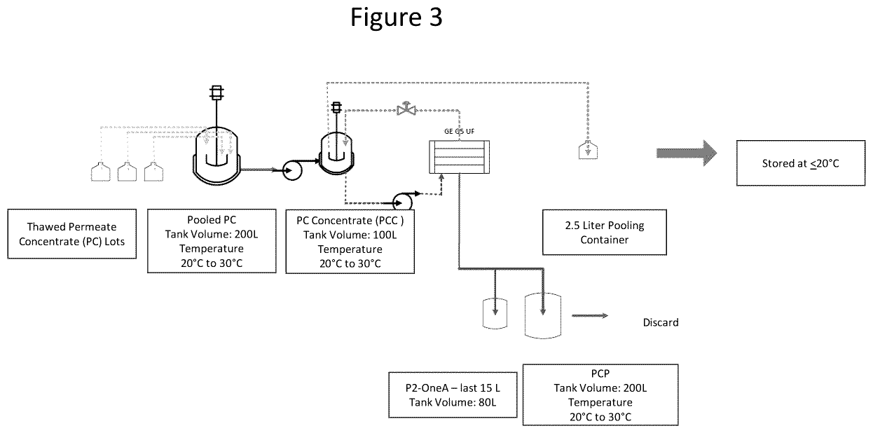

In some embodiments, an additional ultrafiltration is performed through a smaller membrane than the initial a membrane with molecular weight cut-off of ≤50,000 Dalton. In some embodiments, the further ultrafiltration is performed with a membrane with a molecular weight cut off of about 2,000-3,000 Dalton. This additional, optionally, filtration step further aids in the overall purity of the HMO product, by assisting in the removal of smaller potentially bioactive and/or immunogenic factors such as microRNAs and exosomes. FIG. 3 shows an embodiment with this additional filtration step.

In one embodiment, the clarified mixture that has undergone at least one, and in some cases two or more rounds of ultrafiltration (or alternative lactase removal means) is further filtered to purify and concentrate human milk oligosaccharides and to reduce the mineral and monosaccharides content.

In some embodiments, filtration can be accomplished using a nanofiltration membrane. In some embodiments, the membrane has a molecular weight cut-off of ≤1,000 Dalton. In some embodiments, the membrane has a molecular weight cut-off of ≤600 Dalton. In yet other embodiments, the membrane has a molecular weight cut-off of about 400 to about 500 Dalton. This additional nanofiltration is a critical step in removing monosaccharides, minerals, particularly calcium, and smaller molecules to produce the final purified HMO composition.

In some embodiments, additional or alternative steps may be taken for the removal of minerals. Such an additional step may include, for example, centrifugation, membrane clarification (≤0.6 micron), or combination of centrifugation and membrane filtration of heated (>40° C.) or refrigerated/frozen and thawing of HMO Concentrate. The collected supernatant or filtrate of these additional or alternative steps, in some embodiments, is concentrated further using a nanofiltration membrane. In some embodiments, the nanofiltration comprises filtration through a membrane with a molecular cut off of ≤600 Dalton. In some embodiments, these additional steps may be performed at any stage of the process, including but not limited to prior to or after pasteurization.

In some embodiments, the physical property of nanofiltration membranes can be modified, such as chemical modification, to selectively concentrate sialylated HMOs, for example, allowing greater efficiency of neutral HMOs removal from HMO concentrate, in instances where concentrated sialylated HMOs are preferred.

In one embodiment, the purified HMO composition is sterilized. The sterilization may be done by any means known in the art. In some embodiments, the purified HMO composition is pasteurized. In some aspects, pasteurization is accomplished at ≥63° C. for a minimum of 30 minutes. Following pasteurization, the composition is cooled to about 25° C. to about 30° C. and clarified through a 0.2 micron filter to remove any residual precipitated material.

### Purified HMO Compositions

Purified HMO compositions of the present invention have substantially reduced levels of lactose and/or minerals. The term “substantially reduced” as it pertains to lactose levels, and as used herein means having a lactose level of ≤5% w/w. In some embodiments, the purified HMO compositions produced by the method described herein comprise about 4.5 to about 8.5 grams of HMO, less than or equal to about 5% w/w of lactose and a mineral composition shown in Table 1:

TABLE 1
EXEMPLARY MINERAL COMPOSITION OF A REDUCED
MINERAL HMO COMPOSITION

| Mineral | Concentration |
|---|---|
| Calcium (Ca) | <1000 mg/100 g |
| Copper (Cu) | <5 mg/100 g |
| Iron (Fe) | <100 mg/g |
| Magnesium (Mg) | <800 mg/100 g |
| Phosphorus (P) | <800 mg/100 g |
| Potassium (K) | <1500 mg/100 g |
| Sodium (Na) | <10 g/100 g |
| Zinc | <100 mg/100 g |

One of skill in the art will understand that in some instances, such as when the purified HMO product is to be formulated as a powder, for example, the reduction of minerals may be less critical. As such, values presented above are provided as an exemplary formulation only, and in particular an exemplary liquid formulation, although there is no reason this formulation could not be powdered.

In some embodiments, the purified HMO composition comprises from about 0.5% to about 7.5%. In some embodiments, the purified HMO composition comprises from about 1.0% to about 2.0%. In some embodiments, the purified HMO composition comprises from about 2.0% to about 4.0%. In some embodiments, the purified HMO composition comprises from about 4.0% to about 5.0%. In some embodiments, the purified HMO composition comprises from about 5.0% to about 7.5%.

In some embodiments, the purified HMO composition comprises an osmolality of less than about 2000 mOsm/kg. In some embodiments, the purified HMO composition comprises less than or equal to about 10% w/w of glucose. In some embodiments, the purified HMO composition made by the methods described herein comprises less than or equal to about 10% w/w of galactose. The presence of the monosaccharides, glucose and galactose are a result of the breakdown of lactose, and as the lactose levels decrease the monosaccharide levels increase. While much of the monosaccharide content may be removed via the same filtration process that removes the minerals and residual lactase, a low level of monosaccharides remains in the purified HMO product. Unlike the disaccharide lactose, however, the presence of these monosaccharides does not present a clinical problem for the vast number of individuals, particularly at these low levels.

Human milk oligosaccharide compositions of the present invention are substantially similar both structurally and functionally to the profile of HMOs observed across the population of whole human milk. That is to say, since the compositions are derived from a pool of donors, rather than an individual donor, the array of HMOs will be more diverse than in any one typical individual. FIG. 5 shows representative chromatograms of pooled human milk (A and B), human milk permeate (C and D) and the purified HMO compositions made by the methods of the present invention (E and F).

One of the biggest variables in HMO diversity derives from the mother's Lewis blood group and specifically whether or not she has an active fucosyltrasferase 2 (FUT2) and/or fucosyltrasferase 3 (FUT3) gene. When there is an active FUT2 gene, an α1-2 linked fucose is produced, whereas fucose residues are α1-4 linked with the FUT3 gene is active. The result of this “secretor status” is, generally, that “secretors” (i.e. those with an active FUT2 gene) produce a much more diverse profile of HMOs dominated by α1-2 linked oligosaccharides, whereas “nonsecretors” (i.e. those without an active FUT2 gene) may comprise a more varied array of, for example α1,-4 linked oligosaccharides (as compared to secretors), but comprise an over decrease in diversity since they are unable to synthesize a major component of the secretor's HMO repertoire.

In some embodiments, pools of milk can be constructed based on, for example secretor status. That is, in some embodiments, it may be beneficial to collect pools of milk from mothers who are secretors separate from pools of milk from moms who are not secretors. The pools of milk from mothers who are secretors will comprise a large percentage of α1-2 linked HMOs and may be useful for promoting gut health, or reducing inflammation, for example. The pools of milk from mothers who are non-secretors will comprise a much more diverse array of α1-4 linked oligosaccharides and may be useful for treatment or prevention of certain gastrointestinal viral infections, including, for example norovirus or rotavirus. In some embodiments, it may be beneficial to ensure that there is a certain proportion of any human milk pool used to make the purified HMO compositions described herein that derives from secretors vs non secretors and vice versa, to ensure the most diverse and representative HMO profile possible. Polymorphisms in FUT2 and FUT3 are merely common examples of polymorphisms that may be used to select donors for particular pools. One of skill in the art will understand that sorting milk pools on the basis of any polymorphism to construct a milk pool with a certain HMO profile can be done for any polymorphism.

A mother may be determined to be a secretor or nonsecretor prior to donation, alternatively or additionally, the mother's secretor status may be obtained during prequalification of the mother as a donor, and/or once the donated milk is received. Screening for secretor status is routine and may be performed by any routine method.

### Uses of Purified HMO Compositions

The purified HMO compositions of the present invention may be added to human milk fortifier compositions, to human milk, to infant formula, non-human milk or the like to increase its nutritional and/or immunologic value. Alternatively, the purified human milk oligosaccharide compositions of the present invention may be formulated into an oral solution for consumption by infants, older children, and adults. In some embodiments, the purified HMO compositions made by the methods herein may be lyophilized or free-dried or otherwise powdered.

Owing to the anti-infective, immunomodulatory and pre-biotic effects of the purified HMO compositions made by the methods described herein, the compositions find use in a wide variety of biological and clinical contexts. Such uses include, but are not limited to, as an antiadhesive antimicrobial, as a modulator of intestinal epithelial cell response, as an immune modulator, and/or a protectant against necrotizing enterocolitis (NEC).

Purified human milk oligosaccharide compositions of the present invention are useful in positively altering the microbiota of the human mucosa (e.g. the gastrointestinal or urogenital tract) affecting the generation of anti-inflammatory mediators, and or preventing adhesion of pathogenic bacteria on the intestinal epithelial surface.

The present invention provides a method of administering a purified HMO composition made according a method described herein to a subject. In some embodiments, the subject is a human preterm or full term infant. In some embodiments, the subject is a child. In some embodiments the subject is an adult. In some embodiments, the composition is administered topically, orally, or rectally. In some embodiments, the composition is administered orally via a feeding tube.

In some embodiments, the purified HMO composition of the present invention may be administered before during or after treatment with another active agent. For example, the purified HMO composition may be administered as part of an antibiotic, antiviral, antifungal, and/or probiotic course of therapy and in combination with antibiotic and probiotic agents. In one embodiment, the purified HMO composition may be administered in connection with chemotherapy or radiation.

In some embodiments, the purified HMO compositions made by the methods described herein have a synergistic effect when administered in combination with antibiotics. In some embodiments, the purified HMO compositions may be administered in conjunction with a fecal transplant or to a subject being administered, to be administered or recently administered a fecal transplant.

The present invention provides methods of treating a subject who has an infection or is at risk of developing an infection comprising administering a purified human milk oligosaccharide composition to the subject. In some embodiments, the symptoms of the infection are caused by bacteria, bacterial toxins, fungi, or viruses. In some embodiments, the subject is a human. In some embodiments, the infection is caused by a bacteria. In some embodiments, the bacteria is Clostridium difficile. In some embodiments, the infection is caused by a virus. In some embodiments, the virus is a norovirus, or a rotavirus. In another embodiment, the virus is a hemorrhagic virus that causes symptoms by inflammatory burst. In some embodiments, the virus is an Ebola virus or other hemorrhagic fever virus. In some embodiments, the subject is a human neonate, infant, child or an adult. In some embodiments, treating comprises ameliorating at least one symptom of the infection. In some embodiments, treating comprises promoting the development of beneficial gut bacteria. In some embodiments, the beneficial gut bacteria are one or more of bifidobacteria, lactobacilli, streptococci or enterococci.

In some embodiments, the purified HMO composition of the present invention may be administered to a subject in need thereof as an anti-inflammatory agent. In some embodiments, the subject in need thereof has an inflammatory condition. In some embodiments, the subject has inflammatory bowel disease. In some embodiments, the subject has colitis. In some embodiments, the subject has ulcerative colitis. In some embodiments, the subject has pouchitis. In some embodiments, the subject has Crohn's disease. In some embodiments, the subject has an autoimmune disease.

In some embodiments, the purified HMO compositions made by the methods of the current invention may be used in connection with a transplant. In some embodiments, the purified HMO composition decreases the risk of rejection or suffering from graft versus host disease in a patient undergoing a transplant. In some embodiments the transplant is a solid organ transplant and in some embodiments, the transplant is a bone marrow transplant.

### EXAMPLES

### Example 1: Human Milk Oligosaccharide Production

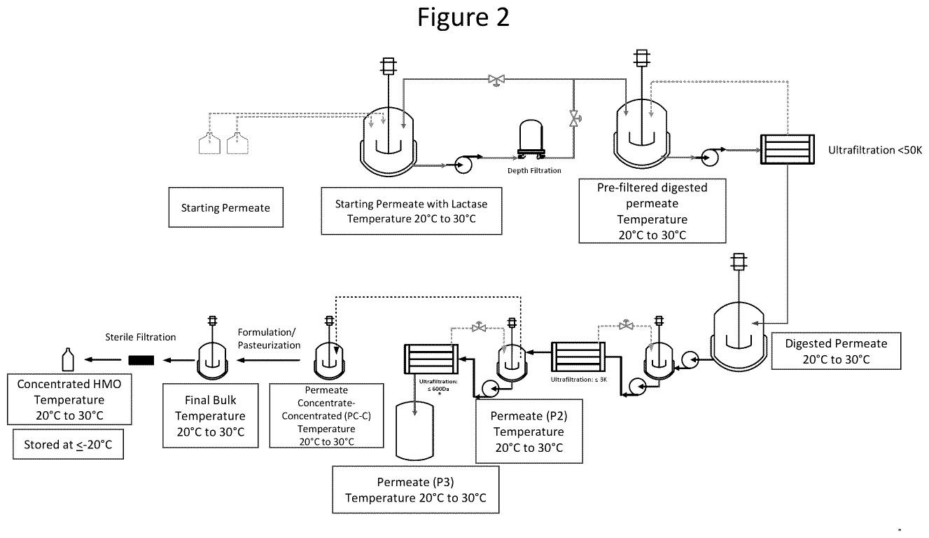

The process for producing a purified HMO composition starts with permeate, as defined above, which was thawed and pooled. The starting permeate temperature was between 23° C.-28° C. The pH of Permeate was adjusted to 4.3 to 4.7 (target 4.5) with the addition of 1N HCl and mixed for about 15 minutes. Permeate was then heated to about 48° C. to about 55° C., preferably 50° C. Lactase enzyme (0.1% w/w) was added to breakdown lactose into monosaccharides and then the solution was mixed for about 60 minutes. The permeate/lactase enzyme mixture was then cooled to about 20° C. to about 30° C., preferably 25° C. and clarified through a depth filter (CUNO60SP). The ultrafiltration membrane (Biomax-50K) was used to remove lactase from the CUNO clarified processing stream. The permeate collected from the Biomax-50K was concentrated using a nanofiltration membrane with nominal 400 to 500 molecular weight cut-off (GE G-5 UF). The G-5 UF concentration process was ended when the permeate concentrate (PC) reached the target of 5% (w/w) of Human Milk Oligosaccharides. The formulated PC was pasteurized and clarified though 0.2 um sterile filters prior to filling. The PC was stored in containers at ≤−20° C., labeled and packaged prior to product shipment. This processes is graphically represented in FIG. 1. An alternative process is shown in FIG. 2.

### Example 2: Processing Permeate Concentrate (PC) to Permeate Concentrate-Concentrate (PC-C)

The frozen permeate concentrate (≥8×, referred to as “PC”) produced according to Example 1 was thawed and pooled while maintaining a temperature range of about 20° C. to about 30° C., preferably 25° C. and mixed for about 10 minutes. The PC was further concentrated by ultrafiltration, for example using GE G-5 UF to achieve the target ≥20× concentrated. The Permeate Concentrate-Concentrate (PC-C) was transferred into milk storage containers and stored in ≤−20° C. freezer for continued processing at a later time. This processes is graphically represented in FIG. 3.

### Example 3: HMO Formulation

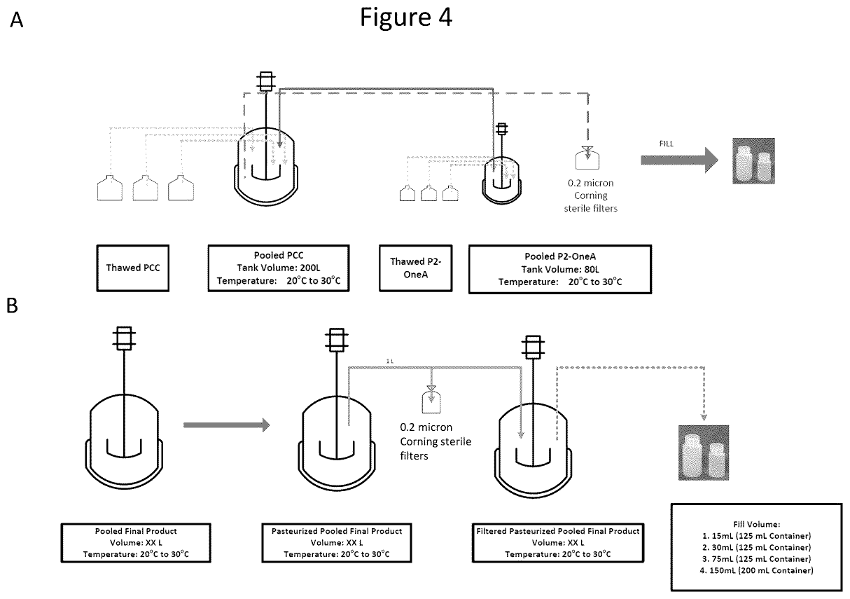

The PC-C was thawed and pooled while maintaining a temperature range of about 20° C. to about 30° C., preferably 25° C. Calculated amount of P2-OneA or purified water was added to PC-C to achieve the final target of 5% w/w HMO. This step is not required if no adjustment of the HMO concentration in the PC-C sample is necessary. This process is graphically represented in FIG. 4 (A).

### Example 4: Final Container Pasteurization and Filtration

If frozen, the concentrated HMO was thawed to about 20° C. to about 30° C., preferably 25° C. It was then pasteurized for about 30 minutes at ≥ 63° C. Following the pasteurization, the concentrated HMO was cooled to a temperature of about 20° C. to about 30° C., preferably 25° C. for clarification through 0.2 micron sterile filters then stored at about 2° C. to about 8° C. A representative sample was taken for visual inspection, total HMO calculation, pH, osmolality, mineral, and sugar analysis.

When the total HMOs results were available, the fill volume was calculated based on the total HMO results in order to achieve the targeted HMO range for each dose.

When HMO results were completed and labels were created, the product was removed from the freezer and transferred to an ISO 8 cleanroom. A label was affixed to each bottle, and each labeled bottled was placed in an airtight bag or an airtight tamper resistant bottle and placed in a crate. Once a crate was complete, the crate was double-bagged and returned back to the freezer at ≤−20° C. until the product is ready for shipment. This process is graphically represented in FIG. 4 (B).

### Example 5: Purified Human Milk Oligosaccharide (HMO) Finished Good Specification

Expiration & Storage: The expiration date was one year from date of pasteurization, minus one day; Storage was frozen at −20° C. or colder.

One representative sample was taken from one of the sterile filter container during the clarification step through 0.2 microns filters. The sample was used for visual inspection, pH, osmolality, sugar profile, mineral content and total HMO calculation. The results of that testing are summarized in Table 2:

TABLE 2
QUALITY CONTROL TEST RESULTS FOR PURIFIED
HMO COMPOSITION

| Test | | Specification |
|---|---|---|
| Visual Inspection | | Yellowish Liquid, |
| | | may have precipitation |
| pH | | 4.0-6.5 |
| Osmolality | | <2000 mOsm/kg |
| (based on extrapolation of | | |
| serial diluted samples) | | |
| Sugar | Lactose | ≤5% (w/w) |
| Profile | Glucose | ≤10% (w/w) |
| | Galactose | ≤10% (w/w) |
| Mineral | Calcium (Ca) | <1000 mg/100 g |
| Content | Copper (Cu) | <5 mg/100 g |
| | Iron (Fe) | <100 mg/100 g |
| | Magnesium (Mg) | <800 mg/100 g |
| | Phosphorus (P) | <800 mg/100 g |
| | Potassium (K) | <1500 mg/100 g |
| | Sodium (Na) | <10 mg/100 g |
| | Zinc (Zn) | <100 mg/100 g |
| Total | 0.1X Target Dose | 0.53 g to 0.71 g |
| HMOs | 0.2X Target Dose | 1.1 g to 1.4 g |
| | 0.5X Target Dose | 2.7 g to 3.6 g |
| | 1.0X Target Dose | 5.3 g to 7.1 g |

### Bioburden Final Container Release Testing

Representative samples were taken from the filling process. Only one bioburden sample was required for each final bulk lot fill. Example: if one (1) final bulk lot was filled into 0.1× and 0.2× target dose, then only one (1) sample was taken to represent both filled 0.1× and 0.2× target dose. The results of those tests are presented in Table 3.

TABLE 3
BIOBURDEN TESTING OF PURIFIED HMO PRODUCT

| Test | Specification |
|---|---|
| Total Aerobic Plate Count (TAC) | <100 CFU/mL¹ |
| E. coli | <1 CFU/mL² |
| Coliform | <1 CFU/mL² |
| Salmonella | Negative/25 mL by ELFA |

¹If result is ≥100 CFU/mL, initiate an exceptional condition and an additional two (2) samples will be tested. The final reported result is the average of the three samples.
²If result is ≥100 CFU/mL, initiate an exceptional condition and an additional two (2) samples will be tested. The final reported result is the average of the three samples.

### Example 6: Evaluation of Bioavailability and Bioactivity of Purified HMO

An escalating dose controlled initial phase trial in 32 healthy adults between the ages of 18 and 50 was conducted to evaluate the bioavailability and potential effects of the purified HMO composition made by the method of the present invention and described in the preceding examples on the immune system.

Study subjects consumed the purified HMO concentrate made by the methods described in the previous Examples by mouth three times per day for seven consecutive days (days 1-7). Four separate groups of male and female study subjects received purified HMO composition at the following concentrations, 0.1×, 0.2×, 0.5× and 1×, where x represents the total weight of HMO calculated to be given to a 70 kg adult based on the concentration in human milk and the dose given on a per weigh basis to a premature infant. Currently, this amounts to be 0.75 g/kg based on an infant feeding volume of 150 mL/kg/day. As a result, a 70 kg adult receiving 1× would receive 52.5 g of the purified HMO composition made herein.

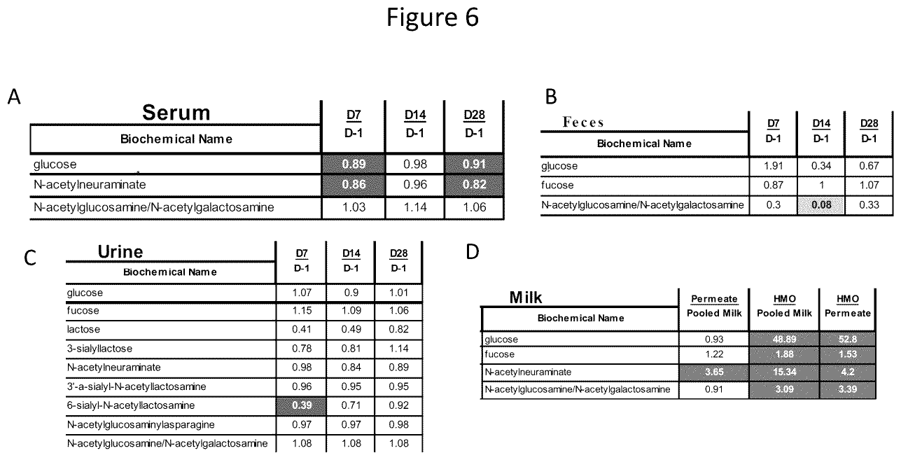

Samples of blood, urine, stool and saliva from all subjects and vaginal swabs from female subjects was taken on days −1 (where day 1 is the first day of ingestion of the purified HMO composition), 7, 14 and 28. Urine, blood and stool were tested for the presence of the parental HMO 3-siallactose as well as the HMO bases, glucose, fucose, N-acetylglucosamine and sialic acid. The parentally HMO 3-siallactose was found in tact only in the urine, suggesting recirculation of the HMO, however, the breakdown products of HMOs are found in all three of urine, blood and stool (FIG. 6), confirming that the orally delivered purified HMO composition is, bioavailable.

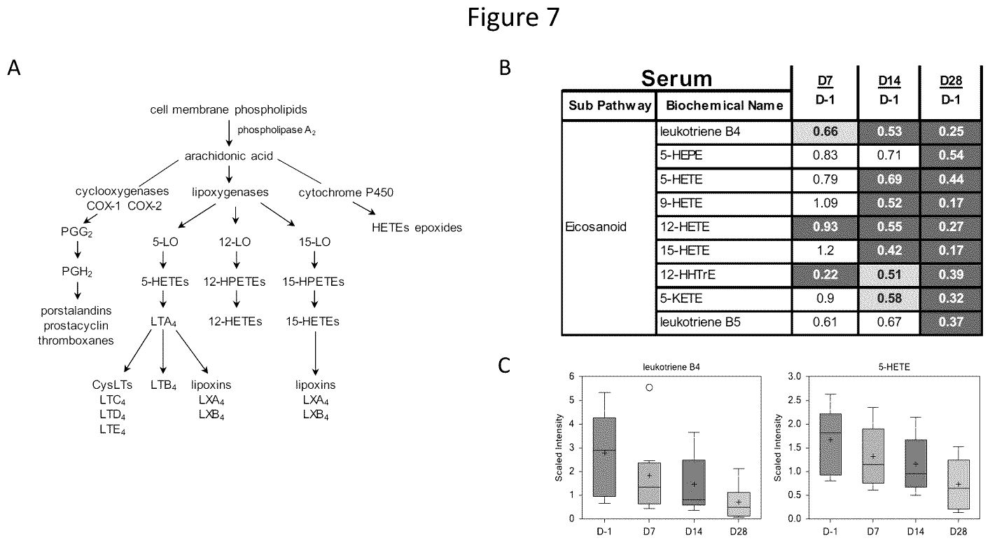

In order to determine if the orally ingested purified HMO composition administered in this study was bioactive, and particularly, whether the purified HMO composition has a physiological effect on systemic markers of inflammation, serum eicosanoids were assayed. Eicosanoids are a diverse family of immune activators that are produced by phospholipase A's action on cell membrane phospholipids (See FIG. 7 (A)) and their elevation in the serum represent an indication of an immune response.

As shown in FIGS. 7 (B) and (C), there was a decreased level of eicosanoids and their metabolites present in the serum of study subjects and this decrease only became more significant over time suggesting that the purified HMO compositions are not only bioavailable but are also bioactive and capable of decreasing the overall inflammatory signature of subjects receiving the composition.

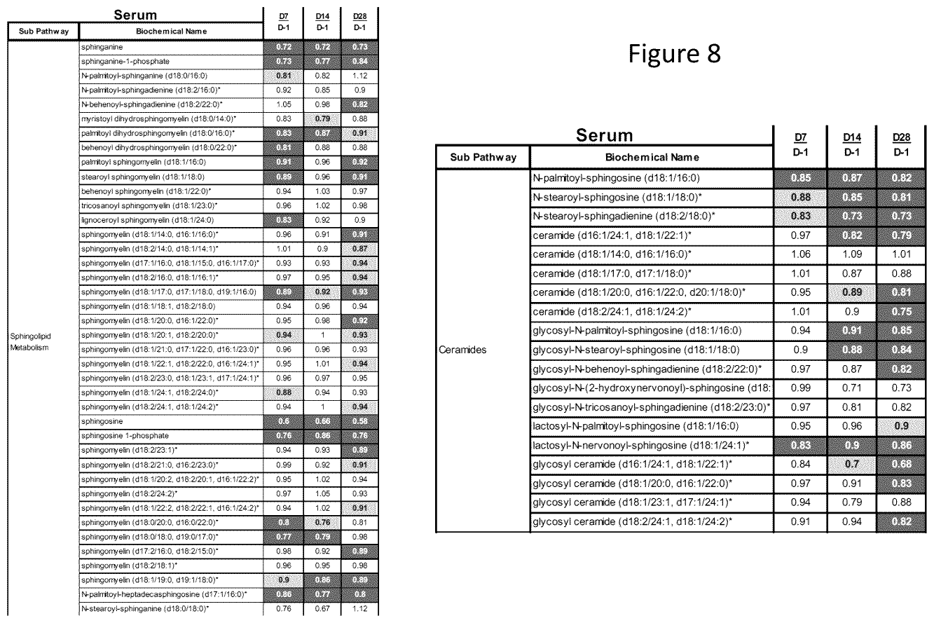

In order to further verify this bioactivity, serum metabolites of sphingolipid metabolism, another marker of inflammation, were also assayed. As shown in FIG. 8, similar to eicosanoids, several sphingolipid metabolites are also reduced over time in subjects receiving the purified HMO compositions made by the methods described here.

Taken together, presented here for the first time is a method to efficiently produce purified HMO compositions, which comprise the full complement of HMOs, with a substantial reduction in lactose and/or mineral content. What is more, this novel purified HMO composition is shown herein to be both bioavailable, as well as bioactive with marked effects on the immune system.
1. Bao Y, Zhu L, Newburg DS. Simultaneous quantification of sialyloligosaccharides from human milk by capillary electrophoresis. Anal Biochem. 2007; 370:206-214.
2. Bode, L. Human milk oligosaccharides: Every baby needs a sugar mama. Glycobiology. 2012 Sep; 22(9): 1147-1162. Published online 2012 Apr 18.
3. 14. Bode, et al. (2016) Nutritional Reviews, 74(10):635-644
4. Chaturvedi P, Warren CD, Altaye M, Morrow AL, Ruiz-Palacios G, Pickering LK, Newburg DS. Fucosylated human milk oligosaccharides vary between individuals and over the course of lactation. Glycobiology. 2001; 11:365-372.
5. Coppa GV, Pierani P, Zampini L, Carloni I, Carlucci A, Gabrielli O. Oligosaccharides in human milk during different phases of lactation. Acta Paediatr. 1999; 88:89-94.
6. Davidson B, Meinzen-Derr JK, Wagner CL, Newburg DS, Morrow AL. Fucosylated oligosaccharides in human milk in relation to gestational age and stage of lactation. Adv Exp Med Biol. 2004; 554:427-430.
7. Gabrielli O, Zampini L, Galeazzi T, Padella L, Santoro L, Peila C, Giuliani F, Bertino E, Fabris C, Coppa GV. Preterm milk oligosaccharides during the first month of lactation. Pediatrics.2011; 128:e1520-1531.
8. German, JB et al. Nestle Nutr Workshop Ser Pediatr Program. 2008. 62:205-18.
9. Grandison, et al (2002) Songklanakarin J. Sci. Technol. 24(Suppl):91-928.
10. Kunz C, Rudloff S, Baier W, Klein N, Strobel S. Oligosaccharides in human milk: Structural, functional, and metabolic aspects. Annu Rev Nutr. 2000; 20:699-722.
11. Kunz C, Rudloff S, Schad W, Braun D. Lactose-derived oligosaccharides in the milk of elephants: Comparison with human milk. Br J Nutr. 1999; 82:391-399.
12. Kunz et al. “Bioactivity of Human Milk Oligosaccharides” page 5 in Food Oligosaccharides: Production, Analysis and Bioactivity, First Edition. Edited by Dr. F. Javier Moreno and Dr. Mar\grave{ }ιa Luz Sanz
13. Morrow Ruiz-Palacios GM, Jiang X, Newburg DS., Human-milk glycans that inhibit pathogen binding protect breast-feeding infants against infectious diarrhea. J. Nutri. 135:1304-07, 2005.
14. Newburg DS, Shen Z, Warren CD. Quantitative analysis of human milk oligosaccharides by capillary electrophoresis. Adv Exp Med Biol. 2000; 478:381-382.
15. Sarney, et al (2000) Biotechnol. Bioeng. 69:461-467
16. Thum, et al. (2015) J Food Comp and Anal., 42:30-37.
17. Ward (2009) Open Glycoscience 2: 9-15
18. Zivkovic, et al (2011) Proc. Natl. Acad. Sci., 108(s1):4653-4658.

1-22. (canceled)

23. A method for preventing necrotizing enterocolitis in a subject in need thereof, the method comprising:
adding a purified human milk oligosaccharide composition to an infant formula to obtain a mixed infant formula, wherein the purified human milk composition comprises: at least 5% human milk oligosaccharides by weight/weight, less than or equal to 5% lactose by weight/weight, and at least ten structurally distinct human milk oligosaccharides; and
administering the mixed infant formula to a premature infant in need thereof.

24. The method of claim 23, wherein the purified human milk oligosaccharide composition comprises as least fifty structurally distinct human milk oligosaccharides.

25. The method of claim 23, wherein the purified human milk oligosaccharide composition comprises at least one neutral human milk oligosaccharide and at least one sialylated human milk oligosaccharide.

26. The method of claim 23, wherein the purified human milk oligosaccharide composition comprises at least one human milk oligosaccharide comprising an α1-2 linked fucose and at least one human milk oligosaccharide comprising α1-4 linked fucose.

27. The method of claim 23, wherein the purified human milk oligosaccharide composition comprises 2′-fucosyllactose (2′FL), 3-fucosyllactose (3′FL), lacto-N-difucohexaose (LNDFH), lacto-N-fucopentaitol I (LNFP I), lacto-N-tetraose (LNT), 3′-sialyllactose (3′SL), 6′-sialyllactose (6′SL), sialyllacto-N-tetraose c (LSTc), sialyllacto-N-tetraose b (LSTb), and disialyllacto-N-tetraose (DSLNT).

28. A method of making a purified human milk oligosaccharide composition comprising:
(a) mixing human milk permeate with lactase under conditions suitable for the digestion of lactose in the human milk permeate to create a permeate/lactase mixture, wherein the human milk permeate is obtained from the ultrafiltration of human skim milk;
(b) removing the lactase from the permeate/lactase mixture; and
(c) filtering the mixture to purify and concentrate human milk oligosaccharides.

29. The method of claim 28, wherein the pH of the permeate is adjusted to a pH of about 4.3 to about 4.7 prior to or concurrent with the addition of the lactase.

30. The method of claim 28, wherein the permeate is heated to a temperature of about 45° C. to about 55° C. prior to or concurrent with the addition of the lactase.

31. The method of claim 28, wherein lactase is added at about 0.1% to about 0.5% weight/weight.

32. The method of claim 28, further comprising cooling the mixture to a temperature of about 20° C. to about 30° C. prior to the removal of lactase.

33. The method of claim 28, further comprising clarifying the lactase digested permeate through a depth filter, wherein the depth filter is about 1 to about 5 micron filter.

34. The method of claim 28, wherein the filtering comprises filtering the mixture of step (b) through a membrane having a pore size of about 50,000 Dalton to create a 50,000 Dalton filtered mixture.

35. The method of claim 34, further comprising filtering the 50,000 Dalton filtered mixture through a membrane having a pore size of about 2,000 Dalton to about 3,000 Dalton to create a 2,000 to 3,000 Dalton filtered mixture.

36. The method of claim 34, further comprising filtering the 50,000 Dalton filtered mixture through a membrane having a pore size of ≤600 Dalton.

37. The method of claim 28, wherein the concentration of human milk oligosaccharides after purification in step (c) is at least 5% weight/weight.

38. The method of claim 28, wherein the concentration of human milk oligosaccharides after purification in step (c) is about 1% to about 5% weight/weight.

39. A purified human milk oligosaccharide (HMO) composition made according to the method of claim 28.

40. A method for decreasing systemic inflammation in a subject in need thereof comprising administering to the subject an effective amount of the purified HMO composition of claim 39.

41. A method for treating or preventing graft versus host disease in a subject in need thereof comprising administering to the subject an effective amount of the purified HMO composition of claim 39.

42. A method for treating or preventing graft versus host disease in a subject in need thereof, the method comprising:
orally administering to the subject an effective amount of a purified human milk oligosaccharide composition, wherein the subject is an adult who has had or will receive a bone marrow or an organ transplant, thereby treating or preventing the graft versus host disease;
wherein the purified human milk composition comprises: at least 5% human milk oligosaccharides by weight/weight, less than or equal to 5% lactose by weight/weight, and at least ten structurally distinct human milk oligosaccharides.
